(19) United States
(12) Patent Application Publication
(10) Pub. No.: US 2010/0114168 A1
(43) Pub. Date: May 6, 2010

(54) ANCHOR WITH NON-THREADED SECURING MECHANISM TO ATTACH AN ELONGATED MEMBER TO A BONE

(75) Inventor: Keith E. Miller, Germantown, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/261,253

(22) Filed: Oct. 30, 2008

(51) Int. Cl. A61B 17/70; A61B 17/04

(52) U.S. Cl. 606/264; 606/246; 606/278; 606/305; 606/308

(57) ABSTRACT

An anchor with a non-threaded securing mechanism for attaching an elongated member to a bone. The anchor includes a receiver, shaft, and securing mechanism. The receiver includes spaced-apart arms that form a channel sized to receive the elongated member. The shaft extends from the receiver and is configured to engage with the bone. The securing mechanism, including a base and an insert, attaches to the receiver to capture the elongated member in the channel. The base is configured to attach to the arms and includes an aperture that receives the insert. The insert extends through the aperture and may contact against the elongated member.

### RELATED APPLICATION

Co-pending U.S. patent application Ser. No. 12/260,823 is directed to an anchor with a two member securing mechanism for attaching an elongated member to a bone.

### BACKGROUND

The present application relates to anchors for attaching an elongated member to a bone, and more particularly to anchors with a non-threaded securing mechanism to capture the elongated member.

Elongated members such as but not limited to rods, wires, and tethers, are used in a variety of different surgical applications. The elongated members are attached to a bone in a patient and may provide a corrective force, support, or positioning for the bone and other adjacent bones. The elongated members are attached to the bone by anchors that include a receiver and a shaft. The receiver is configured to receive the elongated member, and the shaft is configured to attach to the bone. Many previous anchors include a set screw that is threaded onto the receiver to capture the elongated member. The threaded set screw design may include several drawbacks.

One drawback is the set screw may be threaded improperly onto the receiver. This may cause the threads to be stripped on the receiver and/or set screw. The set screw may bind within the receiver preventing further tightening of the set screw to fully engage the elongated member. Alternatively, the set screw is thereafter removed from the receiver but the threads on the receiver are stripped requiring removal and replacement. Another issue may be the false impression that the set screw is fully tightened in the receiver and engaged with the elongated member. At some point thereafter, the improperly threaded set screw loosens in the receiver requiring a revision procedure.

Another drawback is the set screw may cause the arms of the receiver to splay apart. The set screw may apply an outward force on the arms of the receiver as it is threaded onto the receiver. This force may cause the arms to move apart which may cause the set screw to become loosely attached to the receiver. Again, this may result in the set screw not fully engaging the elongated member, and/or the set screw becoming loose which would require correction during a revision procedure.

### SUMMARY

The present application is directed a device to attach an elongated member to a bone in a patient. The device may include

Other aspects of various embodiments of the anchor are also disclosed in the following description. The various aspects may be used alone or in any combination, as is desired.

### BRIEF DESCRIPTION OF THE DRAWINGS

FIG. 1 is a side view of an anchor attaching an elongated member to a bone according to one embodiment.

FIG. 2 is an exploded perspective view of an anchor for attaching an elongated member to a bone according to one embodiment.

FIG. 3 is a perspective view of a base according to one embodiment.

FIG. 4 is a sectional view cut along line IV-IV of FIG. 3 of the base.

FIG. 5 is a sectional view of a base according to one embodiment.

FIG. 6 is a perspective view of an insert according to one embodiment.

FIG. 7 is a sectional view cut along line VII-VII of FIG. 6 of the insert.

FIG. 8 is a sectional view of an insert according to one embodiment.

FIG. 9 is a sectional view of an insert attached to a base according to one embodiment.

FIG. 10 is a side view of an insert attached to a base according to one embodiment.

FIG. 11 is a sectional view of an anchor attaching an elongated member to a bone according to one embodiment.

FIG. 12 is a sectional view of an anchor attaching an elongated member to a bone according to one embodiment.

### DETAILED DESCRIPTION

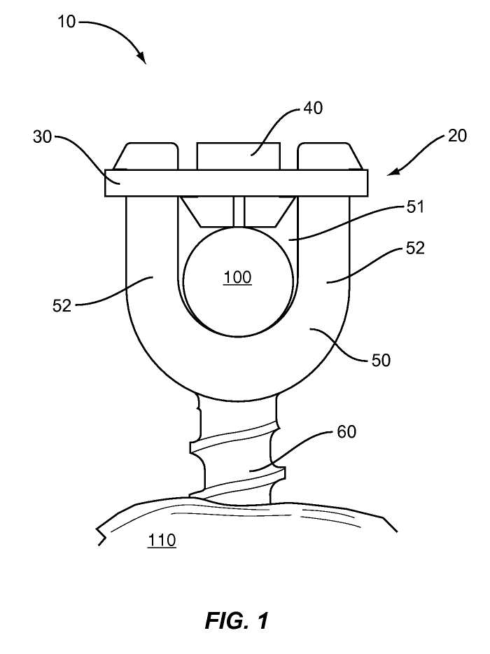

The present application is directed to an anchor with a non-threaded securing mechanism for attaching an elongated member to a bone. FIG. 1 illustrates one embodiment of an anchor 10 securing an elongated member 100 to a bone 110. The anchor 10 includes a receiver 50, shaft 60, and securing mechanism 20. The receiver 50 includes spaced-apart arms 52 that form a channel 51 sized to receive the elongated member 100. The shaft 60 extends from the receiver 50 and is configured to engage with the bone 110. The securing mechanism 20, including a base 30 and an insert 40, attaches to the receiver 50 to capture the elongated member 100 in the channel 51. The base 30 is configured to attach to the arms 52 and includes an aperture 31 that receives the insert 40. The insert 40 extends through the aperture 31 and may contact against the elongated member 100. The securing mechanism 20 does not include threads and attaches to the receiver 50 in a manner to prevent splaying of the arms 52.

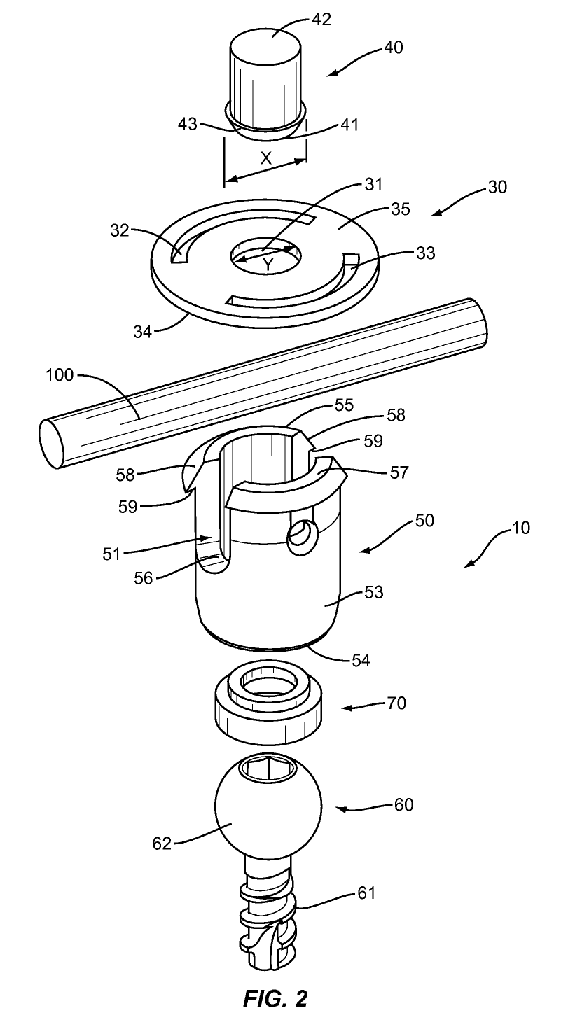

As illustrated in FIG. 2, the receiver 50 includes the arms 52 and a body 53 that form the channel 51. The receiver 50 also includes a first end 54 and a second end 55. A receiving surface 56 of the channel 51 may be shaped to match the size and shape of the elongated member 100. Extensions 57 are positioned on the outer surface of the arms 52. The extensions 57 include a ramped surface 58 and a contact surface 59. The contact surface 59 may be substantially perpendicular to a longitudinal axis of the receiver 50. The extensions 57 may include a width that is greater than the remainder of the arms 52. Further, the width of the extensions 57 increases away from the second end 55 of the receiver 50. FIG. 2 illustrates the extensions 57 positioned at the second end 55 of the arms 52. Extensions 57 may also be positioned along the arms 52 away from the second end 55. Further, multiple extensions 57 may be spaced along the arms 52.

The shaft 60 is configured to attach to the bone 110. The shaft 60 may include threads 61 that engage with the bone 110. Shaft 60 may also include a hook or other shape to engage with the bone 110

The receiver 50 and shaft 60 may be formed as a single piece or as separate pieces that attach together as illustrated in FIG. 2. The two piece construction provides for the receiver 50 to be movable relative to the shaft 60 to accommodate the elongated member 100 at various angular positions. FIG. 2 includes an embodiment with the body 53 of the receiver 50 including a hollow interior sized to receive a head 62 of the shaft 60. A crown 70 may be positioned on the head 62 to facilitate placement and movement of the anchor 60 relative to the receiver 50. U.S. patent application Ser. No. 12/038,572 discloses embodiments of two piece construction and is herein incorporated by reference.

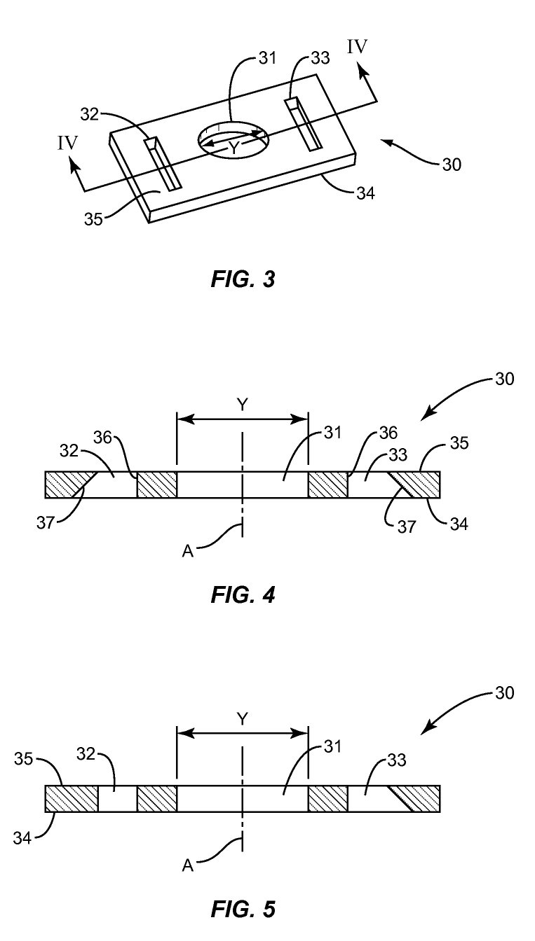

The base 30 includes an aperture 31 with a width Y to receive the insert 40. Base 30 also includes apertures 32, 33 shaped and sized to receive the arms 52 and attach to the receiver 50. The base 30 may include various shapes, such as but not limited to a circular shape as illustrated in FIG. 2 and a rectangular shape as illustrated in FIG. 3. The base 30 may include various thicknesses measured between the first side 34 and the second side 35. Further, the thickness of the base 30 may vary across the length and width.

As illustrated in FIG. 4, the apertures 32, 33 that receive the arms 52 include opposing surfaces 36, 37 when viewed in cross-section. The surfaces 36, 37 are positioned for a width of the apertures 32, 33 to be larger at the first side 34 than at the second side 35. In this specific embodiment, surfaces 36 are substantially parallel to a centerline A of the base 30, and surfaces 37 are positioned at a non-parallel and non-perpendicular angle to the centerline A. If extended outward, the surfaces 36, 37 would form an acute angle and intersect at a point above the second side 35. The decreasing widths of the apertures 32, 33 from the first side 34 towards the second side 35 facilitate attachment to the arms 52 as will be explained in detail below. FIG. 5 includes a base 30 with aperture 32 including a substantially constant width, and aperture 33 including a width that narrows from the first side 34 to the second side 35.

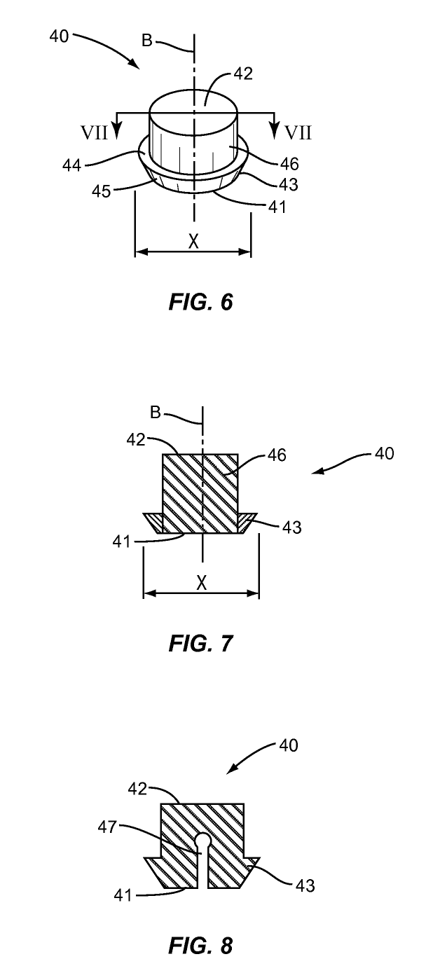

The insert 40 is sized to move along the aperture 31 of the base 30. The insert 40 includes a first side 41 and an opposing second side 42. The insert 40 may be solid, or may include a bore that extends through an interior section from the first side 41 to the second side 42. The insert 40 may also be substantially solid with a cavity that extends inward from one or both sides 41, 42. An extension 43 is positioned between the first and second sides 41, 42. As illustrated in FIG. 6, the extension 43 includes a contact surface 44 that is substantially perpendicular with a longitudinal axis B of the insert 40, and an angled surface 45 that is at a non-parallel and non-perpendicular orientation relative to the longitudinal axis B. The extension 43 may extend continuously around the insert 40, or may include one or more discrete sections.

The extension 43 includes a width X that is greater than the aperture width Y. The extension 43 is constructed to be compressible to fit into the aperture 31. The insert 40 may be constructed in various manners to allow for compression. FIG. 7 includes an embodiment with the extension 43 constructed of a material that compresses while in the aperture 31. A main body 46 of the insert 40 may be constructed of a different material that may or may not compress. If the material does not compress, then the width of the main body 46 is less than the width Y of the aperture 31. In another embodiment, the entire insert 40 is constructed of one or more compressible materials. FIG. 8 includes one or more slots 47 that extend inward from the first end 41 and include opposing sidewalls and an interior end. The slots 47 provide for the extension 43 to compress inward while positioned in the aperture 31. The various embodiments provide for the extension 43 to rebound outward away from the longitudinal axis B when the extension 43 is away from the aperture 31.

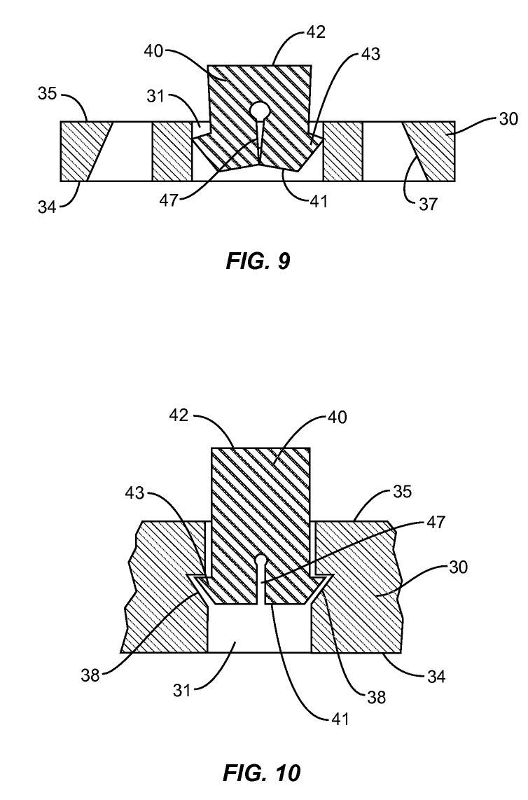

The insert 40 may be operatively connected to the base 30 prior to attachment of the base 30 to the receiver 50. This facilitates attachment of the securing mechanism 20 to the receiver 50 by forming a larger overall structure that may be easier to handle by the surgeon during the surgical procedure. FIG. 9 includes an embodiment with the expansion force of the compressed extension 43 against the surface of the aperture 31 being adequate for attachment. FIG. 10 includes a groove 38 positioned in the sidewall of the aperture 31. The groove 38 is sized to receive the extension 43 to attach the insert 40 to the base 30. The groove 38 may include various shapes and sizes to receive the extension 43. Further, the groove 38 may be located at various locations along the aperture 31 between the first and second sides 34, 35.

The anchor 10 may be constructed of various materials. Examples include but are not limited to titanium, PEEK, polyurethane, polypropylene, or polyethylene, and UHMWPE.

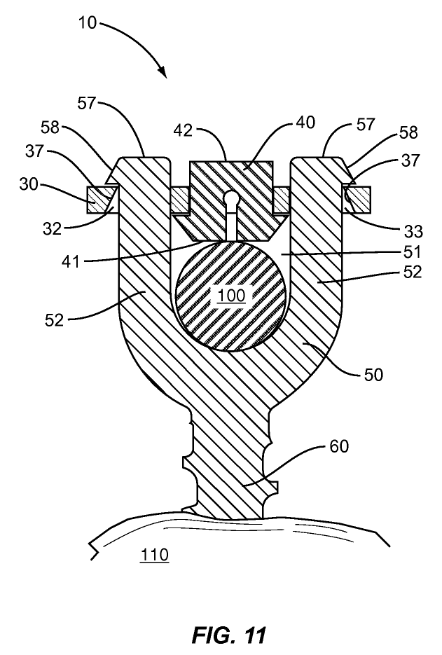

One method of using the anchor 10 includes attaching the receiver 50 and shaft 60 to the bone 110 at the appropriate position to receive the elongated member 100. Once the elongated member 100 is in the channel 51, the securing mechanism 20 is attached to receiver 50 to secure the elongated member 100. Initially, the base 30 is attached to the receiver 50. In one method, the apertures 32, 33 on the base 30 are aligned with the arms 52 on the receiver 50. As illustrated in FIG. 11, angled second surface 37 of apertures 32, 33 are aligned with the angled surfaces 58 of the extensions 57. A downward force is applied to the base 30 to force the base 30 over the arms 52 with the apertures 32, 33 moving over the extensions 57 and the base 30 attaching to the receiver 50. The downward force causes the angled aperture surfaces 37 to slide along the angled extension surfaces 58. The force may cause the arms 52 of the receiver 50 to move together and/or the apertures 32, 33 to elongate to allow the base 30 to move beyond the contact surfaces 59 of the extensions 57. The contact surfaces 59 move over the second side 35 and prevent removal of the base 30 once the second side 35 of the base 30 is moved beyond the angled surfaces 58.

After the base 30 is attached to the receiver 50, the insert 40 is moved towards the elongated member 100. A downward force is applied to the insert 40 to move the extension 43 through the aperture 31. The extension 43 is in a compressed state with a reduced width as it moves through the aperture 31. As illustrated in FIG. 11, the width of the extension 43 increases to a width greater than the aperture 31 after moving beyond the first side 34 of the base 30. This enlarged size prevents the insert 40 from beyond removed from the base 30. As illustrated in FIG. 11, the first side 41 of the insert 40 may contact against the elongated member 100. Alternatively, the first side 41 may be spaced from the elongated member 100. The first side 34 of the base 30 may also spaced away from the elongated member 100 when the base 30 is attached to the receiver 50.

The second side 42 of the insert 40 may be flush with the second side 55 of the receiver 50 upon final deployment. Alternatively, the second side 42 may be recessed below the second side 55, or may extend outward beyond the second side 55.

The force applied to the base 30 and insert 40 may be directly applied by the surgeon. The surgeon may use their finger and/or hand to directly contact against the base 30 and insert. Alternatively, the second sides 35, 42 may include receptacles to receive a tool that is handled by the surgeon and applies the force to the base 30 and/or insert 40.

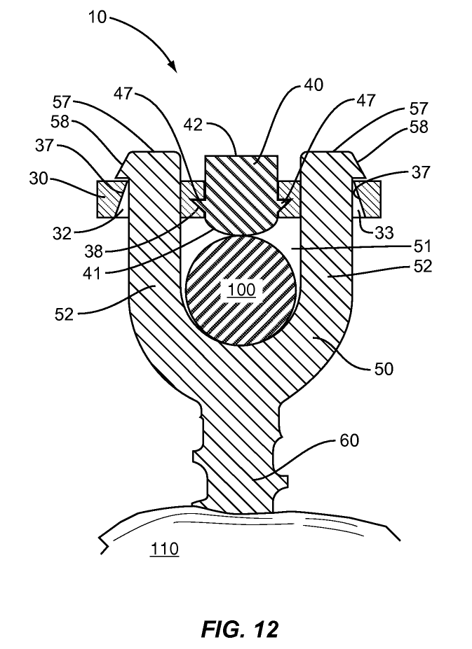

FIG. 12 includes an embodiment with the extensions 43 on the insert 40 positioned away from the first side 41. The insert 40 is attached to the base 30 when the extensions 43 extend into the grooves 38 as illustrated in FIG. 12. In this position, the first side 41 of the insert 40 extends outward into the channel 51 beyond the first side 34 of the base 30. The first side 41 may contact against or be spaced away from the elongated member 100.

The cross-sectional shapes of the insert 40 and aperture 31 may substantially match to facilitate axial movement of the insert 40 relative to the base 30. The insert 40 and aperture 31 may include various shapes, including but not limited to circular, square, rectangular, triangular, oval, and octagonal.

The elongated member 100 may include a variety of configurations, including but not limited to a rod, wire, and tether. The elongated member 100 may be made from a suitably strong rigid material known in the art, such as titanium, or from a semi-rigid material such as PEEK, polyurethane, polypropylene, or polyethylene. The elongated member 100 may include a variety of cross-sectional shapes including but not limited to circular, rectangular, square, and oval. Depending upon the context of use, the elongated member 100 may be linear or non-linear.

The elongated member 100 provides a force to the bone 110. One application includes the elongated member 100 being applied to the spine of a patient with the bone 110 being a vertebral member. In one specific embodiment, the anchor 10 is attached to a pedicle of a vertebral member. Other applications include but are not limited to attachment to the femur, fibula, tibia, humerus, and mandible.

The present invention may be carried out in other specific ways than those herein set forth without departing from the scope and essential characteristics of the invention. Further, the various aspects of the disclosed device and method may be used alone or in any combination, as is desired. The disclosed embodiments are, therefore, to be considered in all respects as illustrative and not restrictive, and all changes coming within the meaning and equivalency range of the appended claims are intended to be embraced therein.

What is claimed is:

1. A device to attach an elongated member to a bone in a patient, the device comprising:
a receiver with a body and a pair of spaced-apart arms that form a channel sized to receive the elongated member, the receiver further including an extension positioned along each of the arms;
a shaft operatively connected to the receiver and configured to attach to the bone; and
a securing mechanism to attach to the receiver and capture the elongated member in the channel, the securing mechanism including a base and an insert, the base including a pair of receiver apertures sized to extend over the arms of the receiver and be captured by the extensions and an intermediate aperture positioned between the receiver apertures that aligns with the channel when the base is attached to the receiver, the insert including first and second ends and being sized to fit within the intermediate aperture and including a compressible extension and a non-threaded exterior;
the insert sized to move along the intermediate aperture with non-rotational movement from a first position with the compressible extension positioned in the intermediate aperture and including a first width with the first end positioned a first distance away from the body of the receiver, and a second position with compressible extension including a larger second width and the first end extending outward beyond the base and being closer to the body than in the first position.

2. The device of claim 1, wherein the first end of the insert is positioned in the intermediate aperture between first and second sides of the base when the insert is in the first position.

3. The device of claim 1, wherein the compressible extension extends outward beyond the intermediate aperture in the second position and the second width of the compressible extension is larger than the intermediate aperture.

4. The device of claim 1, further comprising a slot with opposing sidewalls and an interior end that extends into the first end of the insert, the interior end being spaced farther from the first end of the insert than the compressible extension.

5. The device of claim 1, wherein the intermediate aperture further includes a groove with a width that is larger than the intermediate aperture, the groove including a shape to receive the compressible extension when the insert is in the first position.

6. The device of claim 1, wherein the extension includes a tapered width that increases towards the second end of the insert.

7. The device of claim 6, wherein at least one of the receiver apertures includes a tapered width that is greatest at a first side of the base that faces towards the channel when the base is attached to the receiver and is smallest at a second side.

8. The device of claim 1, wherein the receiver and the shaft are two separate pieces that are operatively attached together with the receiver being movable relative to the shaft.

9. A device to attach an elongated member to a bone in a patient, the device comprising:
a receiver with arms that are spaced apart and form a channel sized to receive the elongated member, each of the arms including an extension;
a shaft that extends outward from the receiver and is configured to attach to the bone;
a base including an intermediate aperture and opposing receiver apertures on each side of the intermediate aperture that are sized to receive the arms of the receiver, the intermediate aperture being aligned with the channel when the receiver apertures are attached to the arms; and
a non-threaded insert with first and second ends and an extension at the first end, the insert sized to fit in the intermediate aperture;
the insert sized to move along the intermediate aperture in a non-rotational manner between a first position with the extension positioned in the intermediate aperture and compressed to a first width and a second position with the extension positioned beyond the base and including a larger second width that is greater than the intermediate aperture and the first end of the insert positioned in the channel to contact against the elongated member.

10. The device of claim 9, wherein the insert includes a polygonal cross-sectional shape.

11. The device of claim 9, wherein the extension includes a contact surface that is perpendicular to a longitudinal axis of the insert and extends continuously around the insert, the contact surface being in contact with a first side of the base when the insert is in the second position

12. The device of claim 9, wherein the insert includes a length with the first end of the insert positioned in the intermediate aperture when the insert is in the first position.

13. The device of claim 9, wherein the insert further comprises a slot with opposing sidewalls and an interior end that extends into the first end of the insert, the interior end being spaced farther from the first end of the insert than the extension.

14. A method of attaching an elongated member to a bone in a patient comprising:
aligning receiver apertures that extend through a base with arms on a receiver that holds the elongated member and is attached to the bone;
moving the base in a non-rotational manner along the arms of the receiver with the arms moving through the receiver apertures;
attaching the base to the arms by moving the base along the arms and beyond extensions on each of the arms; moving in a non-rotational manner an insert along an intermediate aperture in the base and sliding an extension on the insert in a compressed state along the intermediate aperture; and
attaching the insert to the base by moving in the non-rotational manner the insert further along the intermediate aperture and moving the extension beyond the base wherein the extension expands to a width that is greater than the intermediate aperture.

15. The method of claim 14, further comprising positioning the insert partially into the intermediate aperture prior to attaching the base to the arms.

16. The method of claim 14, further comprising contacting the insert against the elongated member.

17. The method of claim 14, wherein moving the base in a non-rotational manner along the arms of the receiver includes sliding a smooth surface of the insert along a smooth surface of the intermediate aperture.
